(19) United States
(12) Patent Application Publication
(10) Pub. No.: US 2005/0214266 A1
(43) Pub. Date: Sep. 29, 2005

(54) Combination of transplantation and oncolytic virus treatment

(75) Inventors: Donald Morris, Calgary (CA); Bradley G. Thompson, Calgary (CA); Matthew C. Coffey, Calgary (CA)

Correspondence Address: FISH & RICHARDSON P.C., PO BOX 1022, MINNEAPOLIS, MN, 55440-1022, US

(73) Assignee: Oncolytics Biotech Inc., Calgary, AB (CA)

(21) Appl. No.: 11/079,008

(22) Filed: Mar. 10, 2005

Related U.S. Application Data
(60) Provisional application No. 60/552,650, filed on Mar. 12, 2004.

(51) Int. Cl. A61K 48/00; C12N 15/86

(52) U.S. Cl. 424/93.21; 435/456

(57) ABSTRACT

Oncolytic viruses can be used to purge cellular compositions to remove undesired neoplastic cells before the cellular compositions are used for transplantation. The present invention relates to the use of a virus to pre-treat a subject prior to delivery into the subject a transplant that has been purged with the same virus. This pre-treatment serves to elicit an immune response in the subject against the virus, thereby protecting the subject from infections by the virus after receiving the transplant, which likely contains infectious viruses.

### RELATED APPLICATION

This application claims the benefit of U.S. Provisional Application No. 60/552,650, filed Mar. 12, 2004, which is herein incorporated by reference.

### TECHNICAL FIELD

This invention relates to pre-treatment with a virus in combination with transplantation.

### REFERENCES

U.S. Pat. No. 6,136,307.
U.S. Patent Application Publication No. 20010048919.
U.S. Patent Application Publication No. 20020006398.
WO 94/18992, published Sep. 1, 1994.
WO 94/25627, published Nov. 10, 1994.
WO 99/08692, published Feb. 25, 1999.
WO 99/18799, published Apr. 6, 2000.
Alain, T., et al. (2002) Reovirus therapy of lymphoid malignancies. Blood. 100(12):4146-4153.
Armitage, J. O. (1989). Bone marrow transplantation in the treatment of patients with lymphoma. Blood 73(7):1749-1758.
Ball, E. D., et al. (1990). Autologous bone marrow transplantation for acute myeloid leukemia using monoclonal antibody-purged bone marrow. Blood 75(5): 1199-1206.
Bar-Eli, N., et al. (1996). Preferential cytotoxic effect of Newcastle disease virus on lymphoma cells. J. Cancer Res. Clin. Oncol. 122(7):409-415.
Bensinger, W. I. (1998). Should we purge? Bone Marrow Transplant 21(2):113-115.
Bergmann, M., et al. (2001). A genetically engineered influenza. A virus with ras-dependent oncolytic properties. Cancer Res. 61(22):8188-8193.
Bezieau, S., et al. (2001). High incidence of N and K-ras activating mutations in multiple myeloma and primary plasma cell leukemia at diagnosis. Hum Mutat. 18(3):212-224.
Bischoff, J. R., et al. (1996). An adenovirus mutant that replicates selectively in p53-deficient human tumor. Science 274(5286):373-376.
Blagosklonny, M. V., and el-Deiry, W. S. (1996). In vitro evaluation of a p53-expressing aenovirus as an anti-cancer drug. Int. J. Cancer 67(3):386-392.
Bos, J. L. (1989). Ras oncogenes in human cancer: a review. Canc. Res. 49(17):4682-4689.
Brasseur, N., et al. (2000). Eradication of multiple myeloma and breast cancer cells by TH9402-mediated photodynamic therapy: implication for clinical ex vivo purging of autologous stem cell transplants. Photochem Photobiol. 72(6):780-787.
Chang, et al. (1992). The E3L gene of vaccinia virus encodes an inhibitor of the interferon-induced, double-stranded RNA-dependent protein kinase. Proc. Natl. Acad Sci USA 89(11):4825-4829.
Chang, H. W., and Jacobs, B. L. (1993). Identification of a conserved motif that is necessary for binding of the vaccinia virus E3L gene products to double-stranded RNA. Virology ˜194(2):537-547.
Chang, et al. (1995). Rescue of vaccinia virus lacking the E3L gene by mutants of E3L. J. Virol. 69(10):6605-6608.
Coffey, M. C., et al. (1998). Reovirus therapy of tumors with activated ras pathway. Science 282(5392): 1332-1334.
Cooper, B. W., et al. (1998). Occult tumor contamination of hematopoietic stem-cell products does not affect clinical outcome of autologous transplantation in patient with metastatic breast cancer. J Clin Oncol. 16(11):3509-3517.
Deisseroth, A. B., et al. (1994). Genetic markers shows that Ph+ cells present in autologous transplants of chronic myelogenous leukemia (CML) contribute to relapse after autologous bone marrow in CML. Blood 83(10):3068-3076.
Eapen, M. (2002). Report on state of the art in blood and marrow transplantation—the IBMTR/ABMTR summary slide with guide. Int Bone Marrow Transplant Registry Autologous Blood Marrow Transplant Registry 9:1-11.
Freedman, A. S., et al. (1990). Autologous bone marrow transplantation in B-cell non-Hodgkin's lymphoma: very low treatment-related mortality in 100 patients in sensitive relapse. J Clin Oncol. 8(5):784-791.
Fueyo, J., et al. (2000). A mutant oncolytic adenovirus targeting the Rb pathway produces anti-glioma effect in vivo. Oncogene 19(1):2-12.
Gorin, N. C., et al. (1990). Autologous bone marrow transplantation for acute myelocytic leukemia in first remission: a European survey of the role of marrow purging. Blood 75(8):1606-1614.
Gorin, N. C. (1992). Cryopreservation and storage of stem cells. In: Areman E M, Deeg H J, Sacher R A, eds. Bone Marrow and Stem Cell Processing: A Manual of Current Techniques. Philadelphia, Pa.: FA Davis 292-362.
Gratwohl, A., et al. (2001). Hematopoietic stem cell transplantation activity in Europe 1999. Bone Marrow Transplant. 27(9):899-916.
Gribben, J. G., et al. (1989). Successful treatment of refractory Hodgkin's disease by high-dose combination chemotherapy and autologous bone marrow transplantation. Blood 73(1):340-344.
Gribben, J. G., et al. (1991). Immunologic purging of marrow assessed by PCR before autologous bone marrow transplantation for B-cell lymphoma. N Engl J. Med. 325(22):1525-1533.
Gulbins, E., et al. (1996). Activation of ras-signaling pathway by the CD40 receptor. J. Immunol. 157(7):2844-2850.
Heise, et al. (2000). Efficacy with a replication-selective adenovirus plus cisplatin-based chemotherapy: dependence on sequencing but not p53 functional status or route of administration. Clin Cancer Res. 6(12):4908-4914.
Heslop, H. E., et al. (1999). Gene marking to assess tumor contamination in stem cell grafts for acute myeloid leukemia. In: Dicke K A, Keating A, eds. Autologous Blood and Marrow Transplantation: Proceedings of the Ninth International Symposium. Arlington, Tex. 513-520.
Kalakonda, N. et al. (2001). Detection of N-Ras codon 61 mutations in subpopulations of tumor cells in multiple myeloma at presentation. Blood 98(5):1555-1560.
Kawagishi-Kobayashi, M., et al. (1997). Regulation of the protein kinase PKR by the vaccinia virus pseudosubstrate inhibitor K3L is dependent on residues conserved between the K3L protein and the PKR substrate eIF2α. Mol Cell Biol. 17(7):4146-4158.
Keeney, M., et al. (1998). Single platform flow cytometric absolute CD34+ cell counts based on the ISHAGE guidelines. International Society of Hematotherapy and Graft Engineering. Cytometry. 34(2):61-70.
Keir, M., et al. (2000). Sensitivity of c-erbB positive cells to a ligand toxin and its utility in purging breast cancer cells from peripheral blood stem cell (PBSC) collections. Stem Cells. 18(6):422-427.
LaCasse, E. C., et al. (1999). Shiga-like toxin-1 receptor on human breast cancer, lymphoma, and myeloma and absence from CD34+ hematopoietic stem cells: implications for ex vivo tumor purging and autologous stem cell transplantation. Blood 94(8):2901-2910.
Lee, P. W., et al. (1981). Protein 61 is the reovirus cell attachment protein. Virology. 108(1):156-163.
Lemoli, R. M., et al. (1991). Autologous bone marrow transplantation in acute myelogenous leukemia: in vitro treatment with myeloid-specific monoclonal antibodies and drugs in combination. Blood 77(8):1829-1836.
Leung, W., et al. (1998). Frequent detection of tumor cells in hematopoietic grafts in neuroblastoma and Ewing's sarcoma. Bone Marrow Transplant. 22(10):971-979.
Liu, P., et al. (1996). Activating mutations of N- and K-ras in multiple myeloma show different clinical associations: analysis of the Eastern Cooperative Oncology Group phase III clinical trial. Blood 88(7):2699-2706.
Meck, M. M., et al. (2001). A virus-directed enzyme prodrug therapy approach to purging neuroblastoma cells from hematopoietic cells using adenovirus encoding rabbit carboxylesterase and CPT-11. Cancer Res. 61(13):5083-5089.
Nemunaitis, J. (1999). Oncolytic viruses. Invest New Drugs 17(4):375-386.
Nielsen, L. L., and Maneval, D. C. (1998). P53 tumor suppressor gene therapy for cancer. Cancer Gene Ther. 5(1):52-63.
Norman, K. L., and Lee, P. W. (2000). Reovirus as a novel oncolytic agent. J. Clin. Invest. 105(8):1035-1038.
Popescu, N. C., et al. (1985). Chromosomal localization of three human ras genes by in situ molecular hybridization. Somat Cell Mol Genet. 11(2): 149-155.
Reichard, K. W., et al. (1992). Newcastle disease virus selectively kills human tumor cells. Surgical Research 52(5):448-453.
Rill, D. R., et al. (1994). Direct demonstration that autologous bone barrow transplantation for solid tumors can return a multiplicity of tumorigenic cells. Blood 84(2):380-383.
Rizzo, J. D. (1998). New summary slides show current trends in BMT. Int Bone Marrow Transplant Registry Autologous Blood Marrow Transplant Registry. 5:4-10.
Romano, et al. (1998). Inhibition of double-stranded RNA-dependent protein kinase PKR by vaccinia virus E3: role of complex formation and the E3 N-terminal domain. Mol Cell Biol. 18(12):7304-7316.
Schattner, E. J. (2000). CD40 ligand in cell pathogenesis and therapy. Leuk Lymphoma 37(5-6):461-472.
Sharp, J. G., et al. (1996). Outcome of a high-dose therapy and autologous transplantation in non-Hodgkin's lymphoma based on the presence of tumor in the marrow or infused hematopoietic harvest. J Clin Oncol. 14(1):214-219.
Sharp, et al. (1998). The vaccinia virus E3L gene product interacts with both the regulatory and the substrate binding regions of PKR: implications for PKR autoregulation. Virology 250(2):302-315.
Smith, R. E., et al. (1969). Polypeptide components of virions, top component and cores of reovirus type 3. Virology. 39(4):791-810.
Spyridonidis, A., et al. (1998). Minimal residual disease in autologous hematopoietic harvests from breast cancer patients. Ann Oncol. 9(8):821-826.
Spyridonidis, A., et al. (1998). Purging of mammary carcinoma cells during ex vivo culture of CD34+hematopoietic progenitor cells with recombinant immunotoxins. Blood 91(5):1820-1827.
Stojdl, D. F., et al. (2000). Exploiting tumor-specific defects in the interferon pathway with a previously unknown oncolytic virus. Nat. Med. 6(7):821-825.
Strong, J. E., et al. (1993). Evidence that the epidermal growth factor receptor on host cells confers reovirus infection efficiency. Virology 197(1):405-411.
Strong, J. E., and Lee, P. W. (1996). The v-erbV oncogene confers enhanced cellular susceptibility to reovirus infection. J. Virol. 70(1):612-616.
Strong, J. E., et al. (1998). The molecular basis of viral oncolysis: usurpation of the ras signaling pathway by reovirus. EMBO J. 17(12):3351-3362.
Sutherland, D. R. et al. (1996). The ISHAGE guidelines for CD34+cell determination by flow cytometry. International Society of Hematotherapy and Graft Engineering. J Hematother. 5(3):213-226.
Thirukkumaran, C. M., et al. (2003). Reovirus oncolysis as a novel purging strategy for autologous stem cell transplantation. Blood 102(1):377-387.
Tricoli, J. V. et al. (1984). Shows TB. Localization of insulin-like growth factor genes to human chromosomes 11 and 12. Nature. 310(5980):784-786.
Tsujino, I., et al. (2001). Postirradiation hyperthermia selectively potentiates the merocyanine 540-sensitized photoinacitvation of small cell lung cancer cells. Photochem Photobiol. 73(2):191-198.
Villeneuve, L. (1999). Ex vivo photodynamic purging in chronic myelogenous leukaemia and other neoplasias with rhodamine derivatives. Biotechnol Appl Biochem. 30(Pt 1):1-17.

Villunger, A., et al. (1998). Functional granulocyte/macrophage colony stimulating factor receptor is constitutively expressed on neo-plastic plasma cells and mediates tumour cell longevity. Br J Haematol. 102(4):1069-1080.
Wallerstein, R., et al. (1990). A phase II study of mitoxantrone, etoposide, and thiotepa with autologous marrow support for patients with relapsed breast cancer. J Clin Oncol. 8(11):1782-1788.
Wiman, K. G. (1998). New p53-based anti-cancer therapeutic strategies. Med Oncol. 15(4):222-228.
Wu, A., et al. (2001). Biological purging of breast cancer cells using an attenuated replication-competent herpes simplex virus in human hematopoietic stem cell transplantation. Cancer Res. 61(7):3009-3015.
Yoon, S. S., et al. (2000). An oncolytic herpes simplex virus type I selectively destroys diffuse liver metastases from colon carcinoma. FASEB J. 14(2):301-311.
Zorn, U., et al. (1994). Induction of cytokines and cytotoxicity against tumor cells by Newcastle disease virus. Cancer Biother. 9(3):225-235.

All of the publications, patents and patent applications cited above or elsewhere in this application are herein incorporated by reference in their entirety to the same extent as if the disclosure of each individual publication, patent application or patent was specifically and individually indicated to be incorporated by reference in its entirety.

### BACKGROUND OF THE INVENTION

Autologous hematopoietic stem cell (ASC) transplantations following high-dose chemotherapy has gained extensive application as a therapeutic modality in several malignancies (Armitage 1989; Freedman, et al. 1990; Ball, et al. 1990; Gribben, et al. 1989; Wallerestein, et al. 1990; Eapen 2002; and Gratwohl, et al. 2001). Globally, the number of autologous blood and marrow transplantations now surpasses the number of allotransplantations (Eapen 2002; Gratwohl, et al. 2001; and Rizzo, 1998). Despite the significant increase in ASC transplantations, controversy still exists as to the contribution of minimal residual disease to the development of relapse after high-dose chemotherapy. Evidence supported by gene marking studies indicates that relapse after high-dose ablative therapy followed by ASC transplantation may be due to contaminating cancer cells within the autograft (Heslop, et al. 1999; Deisseroth, et al. 1994; and Rill, et al. 1994). It has been estimated that more than 30% of all autografts are contaminated with tumor cells, and this number likely will increase with better detection methodology and increasing use of ASC transplantations in patients with advanced disease.

To minimize the number of contaminating tumor cells, a variety of purging techniques have been used to rid the graft of residual tumor cells. The ideal purging technique should preferentially destroy contaminating tumor cells while preserving the number and function of the collected stem cells. Widely cited purging methods of autografts include the use of ex vivo chemotherapy, tumor targeting monoclonal antibodies linked with toxins or selected on immunocolumns, and positive (CD34+) selection (Lemoli, et al. 1991; Leung, et al. 1998). More recently, photodynarnic purging processes (Tsujino, et al. 2001; Brasseur, et al. 2000; and Villeneuve 1999), virus-directed enzyme prodrug therapy (Meck, et al. 2001), receptor-targeted ligand toxins (Keir, et al. 2000; La Casse, et al. 1999), and attenuated replication-competent virus-based purging techniques (Wu, et al. 2001) have been reported. Yet, to date no method has proved 100% successful in depleting autografts of tumor cells in the clinical setting. Although several studies have suggested that graft manipulation is of clinical benefit (Gorin, et al. 1990; Gribben, et al. 1991; Sharp, et al. 1996; and Spyridonidis, et al. 1998), until purging techniques lead to a complete eradication of contaminating tumor cells, it will be unclear whether recurrence of the disease is the result of the contaminating tumor cells or a reflection of a resistant in vivo malignancy, or both.

The effect of minimal residual disease contributing to relapse following ASC transplantation is significant. In three separate studies involving patients with acute myelogenous leukemia (AML), chronic myelogenic leukemia (CML), and neuroblastoma, residual tumor cells in autografts marked by viral vectors containing the neo gene before infusion have been identified in most cases of relapse (Heslop, et al. 1999; Deisseroth, et al. 1994; and Rill, et al. 1994), confirming that tumor cells within the autograft contribute to relapse. AML and CML are not routinely considered for autotransplantation owing to the beneficial graft-versus-leukemic effect of allotransplantation. The hematologic malignancies tested in the present study are all considered potentially treatable with autotransplantation. Malignancies such as chronic lymphocytic leukemia (CLL) are rarely transplanted owing to the large tumor burden found in most patients' AP and peripheral blood. Only highly selected CLL patients are currently offered ASC transplantation. This number would be significantly increased if adequate purging techniques were available.

Oncolytic viruses, which preferentially kill neoplastic cells over normal cells, can be used to purge transplants and reduce the incidence of residual minimal diseases (U.S. Patent Application Publication Nos. 20010048919 and 20020006398). It is possible that the purged transplant may still contain infectious viruses when being transplanted. Although these viruses do not infect normal cells effectively, they may be more active in tumor patients whose immune systems are weakened due to chemotherapy. Furthermore, transplantation patients usually receive immunosuppressive agents, which will diminish the patient's resistance to viral infections. Therefore, it is desirable to protect transplantation patients from viral infections that are resulted from virus-treated transplants.

### SUMMARY OF THE INVENTION

The present invention provides methods of protecting transplantation patients from viral infections that may result from virus-treated transplants. In the present method, the patient is vaccinated with a virus, to develop immunity to the virus, before receiving a transplant that has been purged with the same virus. Since the patient has developed cellular and/or humoral immunity against this virus, the risk of infections by the virus is greatly reduced. For the purpose of vaccination, attenuated viruses, dead viruses, or even viral fragments or proteins can all be used. However, it is preferable that live, infectious viruses are employed since the pre-treatment with infectious oncolytic viruses provides the patient with the additional benefit of virus therapy.

Accordingly, one aspect of the present invention provides a method for transplanting a cellular composition into a mammal, comprising administering an oncolytic virus to the mammal in an amount sufficient to elicit an immune response to the virus in the mammal, and transplanting into the mammal a cellular composition that has been purged with the virus. The cellular composition preferably comprises hematopoietic stem cells, which can be harvested from bone marrow or blood.

The application of this invention is not limited to hematopoietic stem cells. In another embodiment of this invention, the cellular composition comprises any tissue, organ, a combination of different tissues/organs, or any portion of a tissue or an organ. Examples of the tissue or organ include, but are not limited to, liver, kidney, heart, cornea, skin, lung, pancreatic islet cells, and whole blood.

The oncolytic virus is preferably a reovirus. The virus may also be, for example, an adenovirus, herpes simplex virus, vaccinia virus, influenza virus or parapoxvirus orf.

In one embodiment of this invention, the virus is a mutated or modified virus selected from the group consisting of adenovirus, herpes simplex virus, vaccinia virus, influenza virus and parapoxvirus orf. Each of these viruses in the native form has developed a mechanism to inhibit the double-stranded RNA protein kinase (PKR) to facilitate viral protein synthesis which is otherwise inhibited by PKR. These viruses can therefore replicate in any cells regardless of PKR. When these viral PKR inhibitors are mutated or modified, however, the virus is then susceptible to PKR inhibition and does not replicate in normal cells, which have a functional PKR pathway. These mutated or modified viruses can be used to selectively remove ras-activated neoplastic cells because ras-activated neoplastic cells are deficient in PKR function and thus cannot inhibit replication of these viruses.

In another aspect of this invention, the virus selectively kills neoplastic cells by carrying a tumor-suppressor gene. For example, p53 is a cellular tumor suppressor which inhibits uncontrolled proliferation of normal cells. Approximately half of all tumors have functionally impaired p53 and proliferate in an uncontrolled manner. Therefore, a virus which expresses the wild type p53 gene can selectively kill the neoplastic cells which become neoplastic due to inactivation of the p53 gene product.

A similar embodiment involves viral inhibitors of cellular tumor-suppressor genes. Certain viruses encode a protein which inhibits tumor suppressors, thereby allowing viral replication in the cell. By mutating these viral inhibitors, a virus is generated which does not replicate in normal cells due to the presence of tumor suppressors. However, it replicates in neoplastic cells which have lost the tumor suppressors and can be used to selectively kill neoplastic cells in the present invention.

In another embodiment of the invention, an interferon-sensitive virus is used to selectively kill neoplastic cells. An interferon-sensitive virus is inhibited by interferon and does not replicate in a normal cell which has an intact interferon pathway. Since some neoplastic cells have their interferon pathway disrupted, they can be selectively killed by an interferon-sensitive virus. The interferon-sensitive virus is preferably vesicular stomatitis virus (VSV). Interferon can be optionally added along with the interferon-sensitive virus to remove neoplastic cells.

In a preferred embodiment, the mammal suffers from a neoplasm, particularly a ras-mediated neoplasm. For example, the neoplasm may be selected from the group consisting of lung cancer, prostate cancer, colorectal cancer, thyroid cancer, renal cancer, adrenal cancer, liver cancer, pancreatic cancer, breast cancer and central and peripheral nervous system cancer. Preferably, the neoplasm is selected from the group consisting of Hodgkin's disease, multiple myeloma, DLBCL, CLL, Waldenstrom macroglobulinemia, non-Hodgkin's lymphoma, acute myelogenous leukemia, germ cell (testicular) cancers, brain tumors, and breast tumors.

The mammal may receive additional treatment regimens for the neoplasm, such as chemotherapy, radiation therapy, and/or surgery to remove the neoplasm.

In another embodiment of the invention, the virus-treated transplant is frozen and stored in a solution containing DMSO prior to the transplantation. DMSO is routinely used to freeze and store animal cells but it can denature viruses. Therefore, DMSO treatment removes infectious virus from the transplant while preserving the activity of the transplant in the frozen state for a prolonged period of time.

In another embodiment of the present invention, the virus is removed from the virus-treated transplant by subjecting the transplant to anti-virus antibodies which are specific for the particular virus, or a combination of anti-virus antibodies and complement in order to lyse the virus. Alternatively or additionally, anti-virus antibodies which recognize a molecule on the surface of the virus particle may be used to remove the virus particles by immobilizing the antibodies, applying the transplant to the immobilzed antibodies, and collecting the part of the transplant which does not bind to the antibodies.

Similarly, specific antibodies against the particular virus can be further administered to the transplant recipient to eliminate the virus in vivo, or the recipient can be given an immune system stimulant to achieve this purpose.

In another embodiment of the present invention, the virus is removed from the virus-treated transplant by using a gradient which can separate viruses from cells.

Another aspect of the present invention provides a method for treating a neoplasm in a mammal, comprising administering an oncolytic virus capable of killing cells of the neoplasm to the mammal in an amount sufficient to elicit an immune response to the virus in the mammal, and transplanting into the mammal a stem cell composition that has been purged with the virus.

Yet another aspect of the present invention provides a method for performing stem cell transplantation in a mammal, comprising administering an oncolytic virus capable of killing neoplastic cells to the mammal in an amount sufficient to elicit an immune response to the virus in the mammal, and transplanting into the mammal a stem cell composition that has been purged with the virus.

Still another aspect of the present invention provides a method for reducing the risk of infection by an oncolytic virus in a mammal which may result from transplanting into the mammal a cellular composition that has been purged with the virus, comprising pre-treating the mammal with the virus in an amount sufficient to elicit an immune response to the virus in the mammal before transplanting the cellular composition into the mammal.

The details of one or more embodiments of the invention are set forth in the disclosure below. Other features, objects, and advantages of the invention will be apparent from the description, drawings, and claims.

### BRIEF DESCRIPTION OF THE DRAWINGS

FIG. 1. Reovirus effect on stem cells.

(A) Lack of adverse effect on cultured stem cells. CD34⁺ cells were isolated using negative selection columns and cultured in StemSpan medium in the presence or absence of reovirus (40 MOI). Cells were harvested 0, 1, 2, and 5 days after virus infection, and CD34+cells were enumerated by flow cytometry. Error bars indicate the standard deviation of the mean of 3 replicates.

(B) Lack of adverse effect in stem cell progenitor assay. Stem cells (CD34⁺) were selected and treated with reovirus as described. Samples taken at the days indicated were cultured in methylcellulose and scored for CFU-GMs, BFU-Es, and CFU-GEMMs for granulocytes (G)/macrophage (M), erythroids (E) or granulocyte erythroid macrophage megakaryocyte (GEMM). NV indicates no virus; LV, live virus. Error bars indicate the standard deviation of the mean of 3 replicate plates.

(C) Lack of reovirus protein synthesis detected by [³⁵S]-methionine labeling in apheresis product primed with G-CSF. Apheresis product cells were primed with G-CSF and pulse labeled with [35S]-methionine with or without reovirus treatment (40 MOI). At time points indicated in the figure, cellular proteins were harvested and subjected to SDS-PAGE. Reovirus marker proteins (λ, μ, and σ) are indicated in lane 1. Note the absence of viral protein bands at all time points after virus infection.

FIG. 2. Reovirus and human cancer cell lines.

(A) Effect of reovirus on human cancer cell lines. Monocytic (U937, left panel) and myeloma (RPMI 8226, right panel) cells were infected with reovirus at an MOI of 40 PFU/cell. Cells were harvested at 0, 1, 2, 3, 4, and 7 days after infection, and intact cancer cells were enumerated with propidium iodide using flow cytometry. The values depicted are the means and standard deviations of 4 replicates. The diamonds indicates no virus, and squares indicate live virus.

(B) Reovirus protein synthesis in cancer cell lines. Human monocytic (U937, left panel) and myeloma (RPMI 8226, right panel) cells were infected with reovirus at a MOI of 40 PFUs and pulse labeled with [³⁵S]-methionine for various time points as indicated in the figures. Following labeling, the cells were harvested and lysed, and cellular proteins were subjected to SDS-PAGE. Reovirus proteins (λ, μ, and σ) are shown in lane 1. Reovirus infection and protein synthesis were found in both cell lines.

FIG. 3. Purging effect of reovirus on U937 monocytic cells and RPMI 8226 myeloma cells.

Admixtures of apheresis product cells and U937 monocytic cells (Ai,Bi,Ci) or RPMI 8226 cells (Aii,Bii,Cii) (1%, 0.1%, and 0.01%, respectively) were treated with reovirus (40 MOI). Following 3 days of purging, CD33⁺/CD45⁺ U937 cells or CD138⁺/CD38⁺ myeloma cells were assessed by flow cytometry.

(A) Flow cytometric plots of purged and unpurged samples.

(B) U937 and RPMI 8226 cell numbers in purged and unpurged samples. Arrows indicate the absence of live cells.

(C) Lack of outgrowth of U937 (i) and RPMI 8226 cells (ii) in purged samples following 6 days of incubation. Arrows indicate the absence of regrowth of U937 or RPMI 8226 cells. Error bars represent the standard deviations of the means of 3 replicates.

FIG. 4. Purging effect of reovirus on DLBCL cells.

(A) Cytopathic effect of reovirus on DLBCL cells 48 hours after infection. Purified cells were infected with 40 MOI live virus. Photomicrographs were taken at 48 hours after infection (original magnification, ×200). Significant cytopathic effect indicative of widespread killing was observed in live virus-treated cells, but not in untreated cells.

(B) Flow cytometric analysis of DLBCL following reovirus purging. Apheresis product cells were mixed with DLBCL cells (10%) and purged for 2 days with reovirus. Samples were analyzed using region C and lineage gate D (CD 10⁻CD19⁺) to enumerate λ monoclonal CD10⁻CD19⁺ malignant cells (region C+D). Flow-count beads were included in the CAL region to calculate absolute counts.

(C) Representative histograms of viable DLBCL cells before and after purging with reovirus. Arrow indicates the absence of DLBCL cells.

FIG. 5. Purging effect of reovirus on CLL cells.

(A) Cytopathic effect of reovirus on human CLL cells 72 hours after infection (original magnification, ×200). Purified cells were infected with 40 MOI live virus. Photomicrographs were taken at 72 hours after infection. Significant cytopathic effect was evident in reovirus-treated cells.

(B) Flow cytometric analysis of CLL following reovirus purging. Apheresis product cells were mixed with CLL cells (10%) and purged for 4 to 5 days with reovirus. CLL was detected using a CD5⁺CD19⁺ region (gate A) combined with 5⁺ dim 20⁺ region (gate B) to detect monoclonal λ B cells (gate A+B).

(C) Representative histograms of viable CLL cells for 4 patients following reovirus purging. Arrows indicate that CLL cells for patients 2 and 3 after virus treatment were not detected.

FIG. 6. Purging effect of reovirus on Waldenstrom macroglobulinemia cells.

(A) Cytopathic effect of reovirus on human Waldenstrom macroglobulinemia cells 72 hours after infection. Original magnification, ×200. Purified cells were infected with 40 MOI reovirus. Photomicrographs were taken at 72 hours after infection. Significant cytopathic effect was observed in live virus-treated cells, but not in untreated cells.

(B) Flow cytometric analysis of Waldenstrom macroglobulinemia following reovirus purging. Apheresis product cells were mixed with Waldenstrom macroglobulinemia cells (10%) and purged for 5 days with reovirus. Samples were analyzed using region C and lineage gate D (CD10⁻CD20⁺) to enumerate monoclonal CD10⁻CD20⁺ malignant cells (region C+D). Flow-count beads were included in the CAL region to calculate absolute counts.

(C) Representative histograms of viable Waldenstrom macroglobulinemia cells before and after purging with reovirus. Arrow indicates that Waldenstrom macroglobulinemia cells were not detected.

FIG. 7. Purging effect of reovirus on SLL cells.

(A) Cytopathic effect of reovirus on human SLL cells 72 hours after infection. Purified cells were infected with reovirus (40 MOI), and cells were photographed 72 hours after infection (original magnification, ×200). Cytopathic effect was seen in reovirus-infected cells, but not in uninfected cells.

(B) Flow cytometric analysis of SLL following reovirus purging. Apheresis product cells were mixed with SLL cells (10%) and purged for 5 days with reovirus. Samples were analyzed using flow cytometry. Dim CD5⁺CD19⁺CD20⁺ B cells were gated using 2 regions (A and B) and assessed for clonality. The λ-positive SLL cells were clearly distinguished from the normal polyclonal B cells.

(C) Representative histograms of viable SLL cells before and after purging with reovirus. The arrow indicates that SLL cells were not detected.

FIG. 8. Purging of Burkitt lymphoma, follicular lymphoma, and multiple myeloma cells.

(A) Representative histograms of viable Burkitt lymphoma cells analyzed by flow cytometry following reovirus purging. Apheresis product cells were mixed with Burkitt lymphoma cells (1%) and purged for 3 days with reovirus. Samples were analyzed by flow cytometry using Dim CD10⁺CD19⁺B cells. The λ-positive Burkitt lymphoma cells were detected in both purged and unpurged samples.

(B) Representative histograms of viable follicular lymphoma cells analyzed by flow cytometry following reovirus purging. Apheresis product cells were mixed with follicular lymphoma cells (1%) and purged for 3 days with reovirus. Samples were analyzed by flow cytometry using Dim CD10⁺CD19⁺CD20⁺ B cells. The λ-positive follicular lymphoma cells were detected in both purged and unpurged samples.

(C) Representative histograms of viable multiple myeloma cells analyzed by flow cytometry following reovirus purging. Apheresis product cells were mixed with multiple myeloma cells (5%) and purged for 5 days with reovirus. Samples were analyzed by flow cytometry using CD138⁺CD38⁺ dimCD45⁺ cells. More than 50% of myeloma cells were purged by reovirus.

### DETAILED DESCRIPTION OF THE INVENTION

The present invention provides methods of protecting transplantation patients from viral infections that may result from virus-treated transplants. In the present method, the patient is vaccinated with a virus to develop immunity to the virus before receiving a transplant that has been purged with the same virus. A variety of viruses are useful in this invention; for instance, reovirus which selectively kills ras-activated neoplastic cells. Subjects with ras-activated neoplasms may also be pre-treated with a virus in which the viral inhibitor of double-stranded protein kinase (PKR) is mutated or modified. If the subject is suspected of containing p53-deficient tumor cells, it can be pre-treated with a virus expressing the p53 tumor-suppressor gene, which induces apoptosis in tumor cells with functional impairment in the p53 gene product (Wiman 1998; Nielsen, et al. 1998). Vesicular stomatitis virus (VSV) or other interferon-sensitive viruses can be used in the presence of interferon for the treatment of neoplastic cells with a disrupted interferon pathway.

Other examples of viruses useful in this invention include, without being limited to, vaccinia virus, influenza virus, varicella virus, measles virus, herpes virus and Newcastle Disease Virus, which were reported to be associated with tumor regression or death (Nemunaitis 1999). However, this invention encompasses the use of any virus which is capable of selectively killing neoplastic cells.

Prior to describing the invention in further detail, the terms used in this description are defined as follows unless otherwise indicated.

Definitions

“Virus” refers to any virus, whether in the native form, attenuated or modified. Modified viruses include chemically modified viruses or recombinantly modified viruses. A recombinantly modified virus may be a mutated virus, a recombinant virus or a reasserted virus. A mutated virus is a virus in which the viral genome has been mutated, namely having nucleotide insertions, deletions and/or substitutions. A recombinant virus is a virus having coat proteins from different subtypes, usually prepared by co-infecting a cell with more than one subtype of the virus, resulting in viruses which are enveloped by coat proteins encoded by different subtypes. A reasserted virus is a multi-segment virus in which the segments have been reassorted, usually by co-infecting a cell with more than one subtype of this virus so that the segments from different subtypes mix and match in the cell.

An “oncolytic virus” is a virus capable of selectively killing neoplastic cells.

“Neoplastic cells”, also known as “cells with a proliferative disorder”, refer to cells which proliferate without the normal growth inhibition properties. A new growth comprising neoplastic cells is a neoplasm or tumor. A neoplasm is an abnormal tissue growth, generally forming a distinct mass, which grows by cellular proliferation more rapidly than normal tissue growth. Neoplasms may show partial or total lack of structural organization and functional coordination with normal tissue. As used herein, a neoplasm is intended to encompass hematopoietic neoplasms as well as solid neoplasms.

A neoplasm may be benign (benign tumor) or malignant (malignant tumor or cancer). Malignant tumors can be broadly classified into three major types. Malignant neoplasms arising from epithelial structures are called carcinomas; malignant neoplasms that originate from connective tissues such as muscle, cartilage, fat or bone are called sarcomas; and malignant tumors affecting hematopoietic structures (structures pertaining to the formation of blood cells) including components of the immune system, are called leukemias and lymphomas. Other neoplasms include, but are not limited to, neurofibromatosis.

“Ras-activated neoplastic cells” or “ras-mediated neoplastic cells” refer to cells which proliferate at an abnormally high rate due to, at least in part, activation of the ras pathway. The ras pathway may be activated by way of ras gene structural mutation, elevated level of ras gene expression, elevated stability of the ras gene message, or any mutation or other mechanism which leads to the activation of ras or a factor or factors downstream or upstream from ras in the ras pathway, thereby increasing the ras pathway activity. For example, activation of EGF receptor, PDGF receptor or Sos results in activation of the ras pathway. Ras-mediated neoplastic cells include, but are not limited to, ras-mediated cancer cells, which are cells proliferating in a malignant manner due to activation of the ras pathway.

“Cellular composition” means a composition comprising cells. The composition may contain non-cellular matter. For example, whole blood is a cellular composition which contains plasma, platelets, hormones and other non-cellular matter in addition to cells such as erythrocytes and leukocytes. A cellular composition may contain cells of various types, origin or organization. For example, tissues and organs which contain different cell types arranged in defined structures are considered cellular compositions.

A “mixed cellular composition” is a cellular composition containing at least two kinds of cells. Typically, the mixed cellular composition contains both normal cells and neoplastic cells. It is preferable that most of the cells in the cellular composition are dividing cells, and the virus selectively kills neoplastic cells but leaves other dividing cells essentially intact.

A cellular composition “suspected of containing neoplastic cells” is a cellular composition which may contain neoplastic cells. For example, any autograft obtained from a subject bearing a neoplasm may contain neoplastic cells. A cell culture which has been in culture for a considerable amount of time may contain spontaneous by neoplastic cells.

A “purged” cellular composition or transplant refers to a cellular composition or transplant that has been treated in order to remove contaminants.

“Substantial killing” means a decrease of at least about 20% in viability of the target neoplastic cells. The viability can be determined by a viable cell count of the treated cells, and the extent of decrease can be determined by comparing the number of viable cells in the treated cells to that in the untreated cells, or by comparing the viable cell count before and after virus treatment. The decrease in viability is preferably at least about 50%, more preferably at least about 70%, still more preferably at least about 80%, and most preferably at least about 90%.

The neoplastic cells may be killed in various manners. For example, they may be lysed by a virus which is capable of lytic infection of neoplastic cells (oncolysis). The neoplastic cells may undergo apoptosis which is induced directly or indirectly by the virus. The cells may also, although less preferably, be killed by the immune system which has been activated by the virus. For example, the virus may induce cytokine production, which activates the natural killer cells, which in turn selectively kills neoplastic cells (Zorn, et al. 1994).

A “replication competent” virus is a virus which is capable of replicating in at least one cell type. As opposed to a replication competent virus, a “replication incompetent virus” contains a mutation in a region of its genome which is essential for its replication, and hence is not capable of replicating in any cell.

An “interferon-sensitive virus” is a virus which does not replicate in or kill normal cells in the presence of interferon. A normal cell is a cell which is not neoplastic as defined above. To test whether a virus is interferon sensitive, a culture of normal cells may be incubated with the virus in the presence of varying concentrations of interferon, and the survival rate of the cells is determined according to well-known methods in the art. A virus is interferon sensitive if less than 20%, preferably less than 10%, of the normal cells is killed at a high concentration of interferon (e.g., 100 units per ml).

“Resistance” of cells to viral infection means that infection of the cells with the virus does not result in significant viral production or yield.

As used herein, “transplanting” a cellular composition means placing the cellular composition into the body of a recipient. The cellular composition may be syngeneic, allogeneic, or xenogeneic to the recipient. Therefore, the transplantation may or may not be autologous, but it is preferably autologous.

As used herein, a “transplant recipient” is a mammal which receives a transplantation of cellular compositions. Preferably the recipient is a human, and more preferably the recipient is a human who is receiving transplantation in the treatment of cancer.

Method

The present invention relates to the use of a virus to pre-treat a subject prior to delivery into the subject a transplant that has been purged with the same virus. This pre-treatment serves to elicit an immune response in the subject against the virus, thereby protecting the subject from infections by the virus after receiving the transplant, which likely contains infectious viruses. A variety of viruses may be used in this method, each one of which is selective for a neoplasm or a group of neoplasms. Although reovirus is used as an example below, a person of ordinary skill in the art can follow the instructions herein and practice the present invention by using viruses other than reovirus.

1. Reovirus

Reovirus selectively lyses ras activated neoplastic cells in vitro, in vivo and ex vivo (Coffey, et al., 1998; WO 99/08692). Normally, cells are not susceptible to reovirus infection. However, if the ras pathway is activated, reovirus can successfully replicate in the cells and eventually results in lysis of the host cells. For example, when reovirus-resistant NIH 3T3 cells were transformed with activated Ras or Sos, a protein which activates Ras, reovirus infection was enhanced (Strong, et al. 1998). Similarly, mouse fibroblasts that are resistant to reovirus infection became susceptible after transfection with the EGF receptor gene or the v-erbB oncogene (Strong, et al. 1993; Strong, et al. 1996).

The ras oncogene accounts for a large number of tumors. Activating mutations of the ras gene itself occur in about 30% of all human tumors (Bos, J. L., 1989), primarily in pancreatic (90%), sporadic colorectal (50%) and lung (40%) carcinomas, and myeloid leukemia (30%). Activation of the factors upstream or downstream of ras in the ras pathway is also associated with tumors. For example, overexpression of HER2/Neu/ErbB2 or the epidermal growth factor (EGF) receptor is common in breast cancer (25-30%), and overexpression of platelet-derived growth factor (PDGF) receptor or EGF receptor is prevalent in gliomas and glioblastomas (40-50%). EGF receptor and PDGF receptor are both known to activate ras upon binding to their respective ligand, and v-erbB encodes a constitutively activated receptor lacking the extracellular domain. Mutations in the N-ras and K-ras genes appear to be common in multiple myeloma (Kalakonda, et al. 2001) and the frequency of Ras mutations can vary between 10% and 40% at presentation and increase up to 70% at relapse (Lui, et al. 1996; Bezieau, et al. 2001). RPMI 8226 myeloma cell proliferation has been shown to be enhanced by GM-CSF induced by the p21-ras/mitogenacitvated protein kinase (MAPK) signaling cascade (Villunger, et al. 1998).

Without being limited to a theory, it seems that reovirus replication is regulated at the translational level (Strong, et al. 1998; Norman, et al. 2000). In untransformed NIH 3T3 cells, early viral transcripts activate the double-stranded RNA-activated protein kinase (PKR), which inhibits translation, thereby inhibiting viral replication. Activated Ras (or an activated element of the ras pathway) presumably inhibits or reverses PKR activation. Therefore, viral protein synthesis proceeds, viral particles are made, and the cells are eventually lysed. Therefore, in addition to ras-activated tumors, other tumors in which PKR is inactivated or deleted can also be selectively killed by reovirus.

In an attempt to simulate minimal residual disease, an ex vivo model system was used. Human apheresis product obtained from patients was admixed with monocytic leukemia and myeloma cell lines and treated with reovirus. Two methods were employed to detect any remaining tumor cells after purging: flow cytometry and tumor regrowth. The results indicate that it is possible to achieve complete purging, up to 1% of tumor burden. Clinically, the amount of tumor burden encountered is frequently less than 0.01% (Cooper, et al. 1998), which is well within the range of successful purging seen in our experiments. Long-term incubation with reovirus suggests that complete purging of cells could be obtained at a 1% tumor burden.

Interestingly, reovirus oncolysis was detected in all 4 CLL specimens tested, and successful purging was obtained within 4 to 5 days at 1% or 10% contamination in 3 of the specimens tested. Sensitivity of reovirus to CLLs has recently been observed (Alain, et al. 2002). Mutations in the Ras gene itself are rare in leukemias and lymphomas. However, both the K-ras protooncogene and the insulin-like growth factor 1 receptor-encoding gene are found on chromosome 12, and trisomy of chromosome 12 is a common occurrence in CLL (Popescu, et al. 1985; Tricoli, et al. 1984). It is possible that the activation of signaling pathways in CLL is autocrine in nature, as both CD40 receptor and its ligand CD154 are expressed in these cells (Gulbins, et al. 1996; Schattner 2000). In addition to DLBCL and CLL purging, we demonstrated complete reovirus purging of small lymphocytic lymphoma and Waldenstrom macroglobulinemia within 5 days. Significant purging of a T-cell lymphoma was also observed at 3 days after purging (data not presented).

Three lines of evidence indicate that the oncolytic property of reovirus did not affect stem cells. (1) [35S]-methionine and SDS-PAGE analysis of apheresis cells incubated in the presence of reovirus for up to 60 hours did not affect normal host protein synthesis. Nor were viral proteins detected. Even with G-CSF stimulation, reovirus did not infect stem cells. (2) Isolated stem cells were cultured in StemSpan medium, followed by exposure to reovirus at an MOI of 40. Reovirus did not affect the stem cell population and terminal differentiation. (3) The inability of reovirus to affect colony formation was demonstrated by plating stem cells that had been incubated with reovirus for up to 5 days in a methylcellulose-based medium. Colony formation in methylcellulose increased with prolonged incubation irrespective of whether they had been exposed to reovirus or not.

Thus, reovirus can be used to purge transplants, particularly hematopoietic cells, of contaminating neoplastic cells. To reduce the risk that the transplant recipient may be infected by the virus used to purge the transplant, the recipient can be vaccinated by using the virus, to develop immunity against the virus, prior to the transplantation.

In addition, it may be desired to remove the virus prior to using the virus-treated transplant. For example, reovirus is not associated with any known disease, but it may be more infectious to cancer patients whose immune systems are weakened due to chemotherapy. Accordingly, in another embodiment of this invention, the transplants which have been treated with a virus are frozen in a solution containing DMSO and thawed prior to transplantation. While DMSO is routinely used to freeze and store animal cells, it denatures viruses, thereby removing infectious virus from the stem cell preparation. This further reduces the risk that the virus may cause undesired infections when it is introduced into the transplant recipient via stem cell transplantation.

In another embodiment, the virus-treated cell compositions are treated with specific antibodies against the particular virus or a combination of the specific antibodies and complements in order to inactivate or lyse the virus. Alternatively or additionally, specific antibodies which recognize a molecule on the surface of the particular virus may be used to remove the virus particles from the virus-treated cellular composition. Thus, the antibodies are immobilized to a column, beads, or any other material or device known in the art, the cellular composition is applied to the immobilzed antibodies, and the part of the composition which does not bind to the antibodies is collected according to a procedure suitable for the particular method of immobilization.

Another method which may be used to remove the virus from virus-treated mixture is to subject the mixture to a gradient which separates cells from the virus, and collect the layer that contains only the cells.

In another embodiment, the transplant recipient is given treatments to stimulate the immune system in order to reduce the risk of virus infection. This treatment may be performed prior to, contemporaneously with, or after the transplantation, but is preferably performed prior to the transplantation. As an alternative treatment or in conjunction with the immune system stimulant, the recipient can be given specific antibodies against the particular virus in order to reduce the risk of virus infection.

It is contemplated that the present method will be useful for the treatment of any neoplasm. Of particular interest will be the treatment of Hodgkin's disease, multiple myeloma, non-Hodgkin's lymphoma, acute myelogenous leukemia, germ cell (testicular) cancers, brain tumors, and breast tumors, since high dose chemotherapy and autologous stem cell transplantation have been performed efficiently in patients with these tumors.

Hematopoietic progenitor stem cells can be obtained from the bone marrow of the patient in advance of treatment. Alternatively, in a cancer patient who has been receiving traditional, non-high dose chemotherapy, many stem cells typically appear in the peripheral blood with or without colony stimulating factor priming. Therefore, hematopoietic progenitor stem cell can be obtained from the blood as apheresis product, which can be stored for a long time before being transplanted. The present invention can be applied to stem cell-containing autografts which are harvested from any tissue source, including bone marrow and blood.

2. Other Viruses which Selectively Kill Ras-Activated Neoplastic Cells

Normally, when virus enters a cell, double-stranded RNA Kinase (PKR) is activated and blocks protein synthesis, and the virus cannot replicate in this cell. Some viruses have developed a system to inhibit PKR and facilitate viral protein synthesis as well as viral replication. For example, adenovirus makes a large amount of a small RNA, VA1 RNA. VA1 RNA has extensive secondary structures and binds to PKR in competition with the double-stranded RNA (dsRNA) which normally activates PKR. Since it requires a minimum length of dsRNA to activate PKR, VA1 RNA does not activate PKR. Instead, it sequesters PKR by virtue of its large amount. Consequently, protein synthesis is not blocked and adenovirus can replicate in the cell.

Vaccinia virus encodes two gene products, K3L and E3L, which down-regulate PKR with different mechanisms. The K3L gene product has limited homology with the N-terminal region of eIF-2α, the natural substrate of PKR, and may act as a pseudosubstrate for PKR. The E3L gene product is a dsRNA-binding protein and apparently functions by sequestering activator dsRNAs.

Similarly, herpes simplex virus (HSV) gene γ₁34.5 encodes the gene product infected-cell protein 34.5 (ICP34.5) that can prevent the antiviral effects exerted by PKR. The parapoxvirus orf virus encodes the gene OV20.0L that is involved in blocking PKR activity. Thus, these viruses can successfully infect cells without being inhibited by PKR.

As discussed above, ras-activated neoplastic cells are not subject to protein synthesis inhibition by PKR, because ras inactivates PKR. These cells are therefore susceptible to viral infection even if the virus does not have a PKR inhibitory system. Accordingly, if the PKR inhibitors in adenovirus, vaccinia virus, herpex simplex virus or parapoxvirus orf virus are mutated so as not to block PKR function anymore, the resulting viruses do not infect normal cells due to protein synthesis inhibition by PKR, but they replicate in ras-activated neoplastic cells which lack PKR activities.

Similarly, the delNS1 virus (Bergmann, et al. 2001) is a genetically engineered influenza A virus that can selectively replicate in ras-activated neoplastic cells. The NS1 protein of influenza virus is a virulence factor that overcomes the PKR-mediated antiviral response by the host. NS1 is knocked out in the deINS1 virus, which fails to infect normal cells, presumably due to PKR-mediated inhibition, but replicates successfully in ras-activated neoplastic cells. Therefore, a modified influenza virus in which NS1 is modified or mutated, such as the delNS1 virus, is also useful in the present invention.

Accordingly, a virus that has been modified or mutated such that it does not inhibit PKR function can be used to purge a transplant suspected of having ras-activated neoplastic cells, as well as to pre-treat the transplant recipient before transplantation according to the present invention. The viruses can be modified or mutated according to the known structure-function relationship of the viral PKR inhibitors. For example, since the amino terminal region of E3 protein interacts with the carboxy-terminal region domain of PKR, deletion or point mutation of this domain prevents anti-PKR function (Chang, et al. 1992, 1993, 1995; Sharp, et al. 1998; Romano, et al. 1998). The K3L gene of vaccinia virus encodes pK3, a pseudosubstrate of PKR. There is a loss-of-function mutation within K3L. By either truncating or by placing point mutations within the C-terminal portion of K3L protein, homologous to residues 79 to 83 in eIF-2a abolish PKR inhibitory activity (Kawagishi-Kobayashi, et al. 1997).

3. Viruses Carrying Tumor-Suppressor Genes or Tumor-Suppressor-Related Genes

In another aspect of this invention, the virus selectively kills neoplastic cells by carrying a tumor-suppressor gene. For example, p53 is a cellular tumor suppressor which inhibits uncontrolled proliferation of normal cells. However, approximate half of all tumors have a functionally impaired p53 and proliferate in an uncontrolled manner. Therefore, a virus which expresses the wild type p53 gene can selectively kill the neoplastic cells which become neoplastic due to inactivation of the p53 gene product. Such a virus has been constructed and shown to induce apoptosis in cancer cells that express mutant p53 (Blagosklonny, et al. 1996).

A similar approach involves viral inhibitors of tumor suppressors. For example, certain adenovirus, SV40 and human papilloma virus include proteins which inactivate p53, thereby allowing their own replication (Nemunaitis 1999). For adenovirus serotype 5, this protein is a 55 Kd protein encoded by the E1B region. If the E1B region encoding this 55 kd protein is deleted, as in the ONYX-015 virus (Bischoff, et al. 1996; Heise, et al. 2000; WO 94/18992), the 55 kd p53 inhibitor is no longer present. As a result, when ONYX-015 enters a normal cell, p53 functions to suppress cell proliferation as well as viral replication, which relies on the cellular proliferative machinery. Therefore, ONYX-015 does not replicate in normal cells. On the other hand, in neoplastic cells with disrupted p53 function, ONYX-015 can replicate and eventually cause the cell to die. Accordingly, this virus can be used to selectively infect and remove p53-deficient neoplastic cells from a transplant. A person of ordinary skill in the art can also mutate and disrupt the p53 inhibitor gene in adenovirus 5 or other viruses according to established techniques, and the resulting viruses are useful in the present method.

Another example is the Delta24 virus which is a mutant adenovirus carrying a 24 base pair deletion in the E1A region (Fueyo, et al. 2000). This region is responsible for binding to the cellular tumor-suppressor Rb and inhibiting Rb function, thereby allowing the cellular proliferative machinery, and hence virus replication, to proceed in an uncontrolled fashion. Delta24 has a deletion in the Rb binding region and does not bind to Rb. Therefore, replication of the mutant virus is inhibited by Rb in a normal cell. However, if Rb is inactivated and the cell becomes neoplastic, Delta24 is no longer inhibited. Instead, the mutant virus replicates efficiently and lyses the Rb-deficient cell. Again, this virus is selective for neoplastic cells and can be used to pre-treat transplant recipient suspected of having Rb-deficient neoplastic cells.

4. Other Viruses

Vesicular stomatitis virus (VSV) selectively kills neoplastic cells in the presence of interferon. Interferons are circulating factors which bind to cell surface receptors which ultimately lead to both an antiviral response and an induction of growth inhibitory and/or apoptotic signals in the target cells. Although interferons can theoretically be used to inhibit proliferation of tumor cells, this attempt has not been very successful because of tumor-specific mutations of members of the interferon pathway.

However, by disrupting the interferon pathway to avoid growth inhibition exerted by interferon, tumor cells may simultaneously compromise their anti-viral response.

Indeed, it has been shown that VSV, an enveloped, negative-sense RNA virus rapidly replicated in and killed a variety of human tumor cell lines in the presence of interferon, while normal human primary cell cultures were apparently protected by interferon. An intratumoral injection of VSV also reduced tumor burden of nude mice bearing subcutaneous human melanoma xenografts (Stojdl, et al. 2000).

Accordingly, in another embodiment of the present invention, VSV is used to pre-treat the transplant recipient. Moreover, it is contemplated that any other interferon-sensitive virus (WO 99/18799), namely a virus which does not replicate in a normal cell in the presence of interferons, can be used in the same fashion. Such a virus may be identified by growing a culture of normal cells, contacting the culture with the virus of interest in the presence of varying concentrations of interferons, then determining the percentage of cell killing after a period of incubation. Preferably, less than 20% normal cells are killed and more preferably, less than 10% are killed.

It is also possible to take advantage of the fact that some neoplastic cells express high levels of an enzyme and construct a virus which is dependent on this enzyme. For example, ribonucleotide reductase is abundant in liver metastases but scarce in normal liver. Therefore, a herpes simplex virus 1 (HSV-1) mutant which is defective in ribonucleotide reductase expression, hrR3, was shown to replicate in colon carcinoma cells but not normal liver cells (Yoon, et al. 2000).

In addition to the viruses discussed above, a variety of other viruses have been associated with tumor killing, although the underlying mechanism is not always clear. Newcastle disease virus (NDV) replicates preferentially in malignant cells, and the most commonly used strain is 73-T (Reichard, et al. 1992; Zom, et al. 1994; and Bar-Eli, et al, 1996). Clinical antitumor activities wherein NDV reduced tumor burden after intratumor inoculation were also observed in a variety of tumors, including cervical, colorectal, pancreas, gastric, melanoma and renal cancer (WO 94/25627; Nemunaitis 1999). Therefore, NDV can be used to remove neoplastic cells from a mixed cellular composition, as well as to pre-treat the recipient of the purged composition.

Moreover, vaccinia virus propagated in several malignant tumor cell lines. Encephalitis virus was shown to have an oncolytic effect in a mouse sarcoma tumor, but attenuation may be required to reduce its infectivity in normal cells. Tumor regression has been described in tumor patients infected with herpes zoster, hepatitis virus, influenza, varicella, and measles virus (for a review, see Nemunaitis 1999). According to the methods disclosed herein and techniques well known in the art, a skilled artisan can test the ability of these or other viruses to selectively kill neoplastic cells in order to decide which virus can be used to remove neoplastic cells from a mixed cellular composition of interest. The recipient of the purged composition can be pre-treated according to this invention.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.
° C=degree Celsius
hr=hour
min=minute
sec=second
μM=micromolar
mM=millimolar
M=molar
M1=milliliter
μl=microliter
mg=milligram
μg=microgram
AML=acute myelogenous leukemia
AP=apheresis product
ASC=Autologous hematopoietic stem cell
BFU-Es=erythroid burst-forming units
CFU-GEMMs=granulocyte, erythroid, macrophage, megakaryocyte, colony-forming units
CFU-GMs=granulocyte-macrophage colony-forming units
CLL=chronic lymphocytic leukemia
CML=chronic myelogenic leukemia
DLBCL=diffuse large B-cell lymphoma
DMEM=Dulbecco's modified Eagle's medium
FBS=fetal bovine serum
G-CSF=granulocyte colony stimulating factor
MOI=multiplicity of infection
PAGE=polyacrylamide gel electrophoresis
PBS=phosphate buffered saline
SDS=sodium dodecyl sulfate
SLL=small lymphocytic lymphoma
   Materials and Methods
   Cell Lines

Established cell lines were obtained from the American Type Culture Collection (ATCC; Manassas, Va.). U937 (monocytic) and RPMI 8226 (myeloma) cells were maintained in RPMI 1640 medium (Gibco BRL, Burlington, ON, Canada) containing 10% fetal bovine serum (FBS). U937 and RPMI 8226 cells were immunophenotyped by flow cytometry to identify markers suitable for minimal residual disease (MRD) detection. Although U937 is listed by ATCC as a histiocytic lymphoma, its immunophenotype suggests it is monocytic in origin: CD45⁺, CD33⁺, CD13⁺, CD15⁺, CD11b⁺, CD36⁺, CD11c⁺, CD4⁺, CD7⁺, CD19⁻, CD20⁻, CD10⁻, CD3⁻, CD2⁻, CD5⁻, CD34⁻, CDK⁻, and CD⁻. CD33 and CD45 were used for the detection of U937 cells. CD38 and CD138 were used for the identification of RPMI 8226 myeloma cells.

Primary Tumor Cells

Primary tumors for which autotransplantations are performed were obtained from peripheral blood (chronic lymphocytic leukemia (CLL)), from bone marrow (Waldenstrom macroglobulinemia, Burkitt lymphoma, multiple myeloma), from spleen (small lymphocytic lymphoma (SLL)), or from lymph node (diffuse large B-cell lymphoma (DLBCL), follicular lymphoma) samples. Diagnosis was based on histopathology, immunohistochemistry, and immunophenotypic studies. A World Health Organization (WHO) classification protocol was followed for the classification of cases. All procedures were approved by the Human Ethics Committee at the University of Calgary, AB, Canada, and samples that were in excess of that needed for diagnostic purposes were used for experiments.

To obtain single cell suspensions, lymph node and spleen samples were mechanically disrupted in a DAKO medi machine (DAKO Diagnostics Canada, Missisauga, ON) and filtered through a 100-μm mesh. When neutrophils comprised more than 50% of peripheral blood or bone marrow samples, isolation of mononuclear cells using Ficoll-Hypaque was used to obtain single cell suspensions for further experiments. Chronic lymphocytic leukemia, follicular lymphoma, and DLBCL cells were maintained in Iscove modified Dulbecco medium (IMDM; Stem Cell Technologies, Vancouver, BC, Canada) containing 15% FBS and antibiotics (Gibco BRL). All other primary tumor samples were kept in RPMI medium with 15% FBS and antibiotics.

Reovirus

Reovirus serotype 3 (strain Dearing) was propagated in L929 cells grown in suspension in Joklik modified Eagle medium (JMEM; Gibco BRL) containing 5% FBS. Virus purification was performed according to the protocol of Smith, et al. 1969, with the exception that β-mercaptoethanol was omitted from the extraction buffer.

Apheresis Product (AP)

All apheresis products used in the present study were obtained from patients registered at the Tom Baker Cancer Centre, Calgary, AB, Canada, after informed consent in accordance with the local institutional review board (IRB). AP mononuclear cells were washed in phosphate-buffered saline (PBS) prior to culturing in RPMI 1640 medium supplemented with 10% FBS or StemSpan (Stem Cell Technologies) medium for stem cell assays.

Cytopathic Effect

Cells lines grown to subconfluence and purified or enriched primary tumor samples in culture media were infected with reovirus at a multiplicity of infection (MOI) of 40 plaque-forming units (PFU)/cell. To assess cytopathic effect, cells were photographed under a light microscope at 48 or 72 hours after infection.

Radiolabeling of Reovirus-Infected Cells and Preparation of Lysates

Cell lines (U937 and RPMI 8226) were grown to subconfluence and infected with reovirus at an MOI of 40 PFU/cell. To evaluate whether reovirus infects AP cells, cells were cultured in RPMI medium containing 10% FBS in the presence or absence of granulocyte colony-stimulating factor (G-CSF) (10 ng/mL) and infected with reovirus at 40 MOI. At various time points after infection, the medium was replaced with media containing 0.1 μCi/mL (0.0037 MBq/mL) (³⁵S)-methionine. After further incubation for 12 hours at 37° C., the cells were washed in PBS and lysed in lysis buffer containing 1% Triton X-100, 0.5% sodium deoxycholate, and 1 mM EDTA (ethylenediaminetetraacetic acid). The nuclei were then removed by low-speed centrifugation, and the supernatants were stored at −80° C. until use. Radiolabeled lysates were subjected to sodium dodecyl sulfate-polyacrylamide gel electrophoresis (SDS-PAGE) as previously described (Lee, et al. 1981).

Cell Counting using Flow Cytometry

Monocytic (U937) and myeloma (RPMI 8266) cells were cultured in the presence or absence of live virus (40 MOI) for up to 7 days. At 0, 1, 2, 3, 4, and 7 days after virus infection, cells were harvested, and 1 mL cell culture suspension containing approximately 1×10⁶ cells was centrifuged at 750g for 1 minute. The cell pellet was resuspended in 1 mL of 50 μg/mL propidium iodide/RNase/Triton X-100 (Sigma Chemical, St Louis, Mo.), and 100 μL Flow-count beads (Beckman Coulter, Hialeah, Fla.) was added to each tube. Intact cells were enumerated using flow-count beads as an internal calibrator.

CD34⁺ CD45⁺ Cell Enumeration

CD34-phycoerythrin (PE) (581) and CD45-fluorescein isothiocyanate (FITC) (J33) (Beckman-Coulter) antibodies were added to 100 μL diluted AP cells using a reverse pipetting technique to ensure accuracy. Samples were incubated for 10 minutes at room-temperature in the dark. Flow-count beads (100 μL) were added to each tube using the same technique as in “Cell counting using flow cytometry.” Flow cytometric analysis was performed on an EPICS XL flow cytometer (Beckman Coulter) using a modified ISHAGE strategy (Keeney, et al. 1998; Sutherland, et al. 1996). Data from 4 parameters were collected for analysis: forward scatter (FS), log side scatter (LSS), log fluorescence 1 (LFL1), and log fluorescence 2 (LFL2). Acquisition was halted at 100,000 CD45⁺ events. Hematopoietic progenitor cells were identified and counted in 2 histograms (CD45-FITC versus CD34-PE and FS versus LSS) using ISHAGE criteria: dim CD45⁺, bright CD34⁺ form a discrete cell cluster with a larger FS signal than lymphocytes. The use of a known amount of Flow-Count fluorospheres allowed the determination of absolute CD34⁺ cell count directly from the flow cytometer.

Effect of Reovirus on CD34⁺ Stem Cells

Apheresis product cells were depleted of lineage-committed cells using the StemSep immunomagnetic cell separation system (Stem Cell Technologies). The StemSep progenitor enrichment antibody cocktail (Catalog no. 14036; Stem Cell Technologies) was used to enrich for CD34⁺ CD38⁻ cells. The isolated cells were seeded at a density of 2 to 3×10₃/mL in StemSpan SFEM (Stem Cell Technologies) containing 40 μg/mL low-density lipoproteins (LDLs) (Sigma) and purified recombinant human Flt-3 ligand (FL; 100 ng/mL), stem cell factor (SCF; 100 ng/mL), interleukin-3 (IL-3; 20 ng/mL), IL-6 (20 ng/mL), and thrombopoietin (Tpo, 50 ng/mL). Cultures were then incubated in the presence or absence of reovirus (40 MOI) for 5 days at 37° C. in a humidified incubator with 5% CO₂. Cells were harvested at days 1, 2, and 5 and assayed for CD34⁺ and CD45⁺ cells and colony-forming cells.

Colony-forming cells were evaluated by plating 10³ cells in methylcellulose (MethoCult GF H4434; Stem Cell Technologies) to result in a 1:10 (vol/vol) ratio. Plates were scored for erythroid burst-forming units (BFU-Es); granulocyte-macrophage colony-forming units (CFU-GMs); and granulocyte, erythroid, macrophage, megakaryocyte, colony-forming units (CFU-GEMMs) following incubation at 37° C. in a humidified 5% CO₂ incubator for two weeks.

Contamination of Apheresis Product with Cancer Cells

U937 monocytic cells and RPMI 8226 myeloma cells were mixed with apheresis product in RPMI 1640 medium supplemented with 10% FBS to result in concentrations of 1%, 0.1%, and 0.01%. Cell admixtures were either treated with reovirus (40 MOI per total cell population) or left untreated and incubated for 3 days.

On day 0 and day 3 of purging, samples were taken from all admixed cell populations, and intact cancer cell numbers were evaluated using flow cytometry. To ensure reovirus treatment did not affect the stem cell population, admixed cell populations were analyzed for CD34⁺CD45⁺ cell counts following 3 days of reovirus treatment. The efficacy of purging was further evaluated by reculturing a portion of the purged and unpurged admixed cells in the appropriate media for each cancer cell line, and viable cancer cell outgrowth counts were enumerated using flow cytometry after 6 days of incubation.

Reovirus Purging of Primary Human Tumors Contaminating Apheresis Product

AP cells were admixed with tumor samples to result in 10%, 5%, 1%, or 0.1% contamination and treated with 40 MOI reovirus per total cell population (AP+tumor cells). Cell admixtures were incubated in a CO₂ incubator as described previously and analyzed for residual disease by flow cytometry. Minimal residual disease was detected in apheresis samples using 5-color immunophenotyping on a Cytomics FC500 flow cytometer (Beckman Coulter) using the antibodies conjugated to the following fluorochromes: FITC, PE, PE-Texas Red (ECD), PE-cyanin 5.1 (PC5), and PE-cyanin 7 (PC7). Analysis strategies included the use of lineage-gating techniques, aberrant marker expression, and enumeration of 1×10⁶ cells per sample to detect rare events. Disease-free apheresis samples were run in parallel as a negative control to assess background levels.

Effect of Cryopreservation and DMSO on Reovirus Viability

To assess whether exposure to dimethyl sulfoxide (DMSO) and/or the cryopreservation procedure affects reovirus viability, apheresis cells were exposed to reovirus (40 MOI) and incubated in a CO₂ (5%) incubator at 37° C. for 3 days as in purging experiments. This procedure was done to quantitate the amount of virus that would potentially be infused to the patients. Virus-treated and untreated apheresis products were then frozen as per local protocol, DMSO medium (20% DMSO (Edward Life Sciences, Irvine, Calif.), 60% TC199 (Stem Cell Technologies), and 20% albumin (Bayer, Elkhart, Ind.) vol/vol) in a 1:1 ratio (Gorin, N.C. 1992). The DMSO-treated cells were subjected to controlled rate cooling immediately in a cryopreservation system (Planer:KRYO 10 series 11; Planer Products, Middlesex, United Kingdom) and maintained in liquid nitrogen for 2 weeks. Frozen apheresis product cells were thawed in a 37° C. water bath similar to the technique used at bedside. A portion of the thawed apheresis product was washed gently in PBS once and resuspended in RPMI medium. Viral plaque titrations of the thawed products were assayed.

### EXAMPLE 1

### Reovirus Does Not Affect Hematopoietic Progenitors

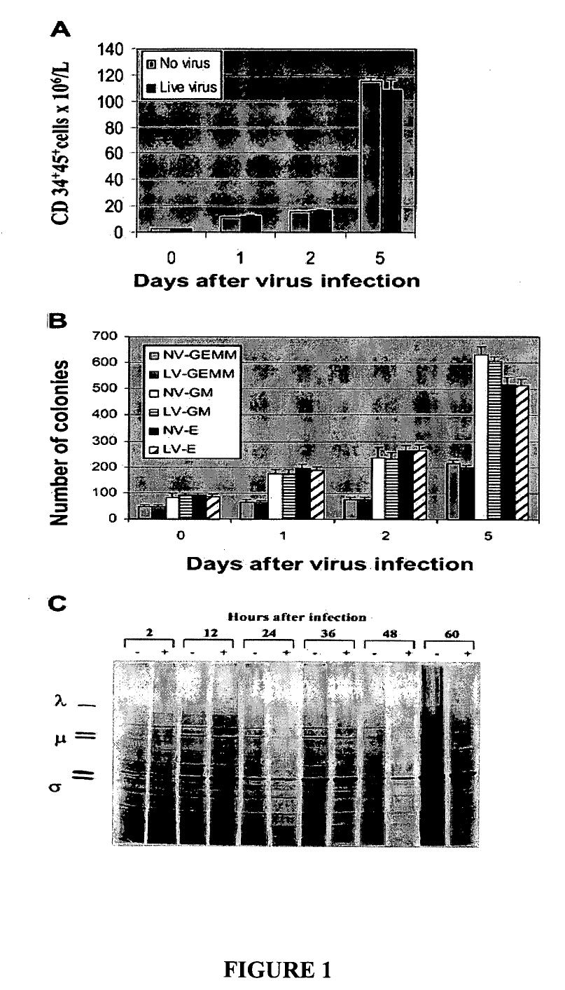

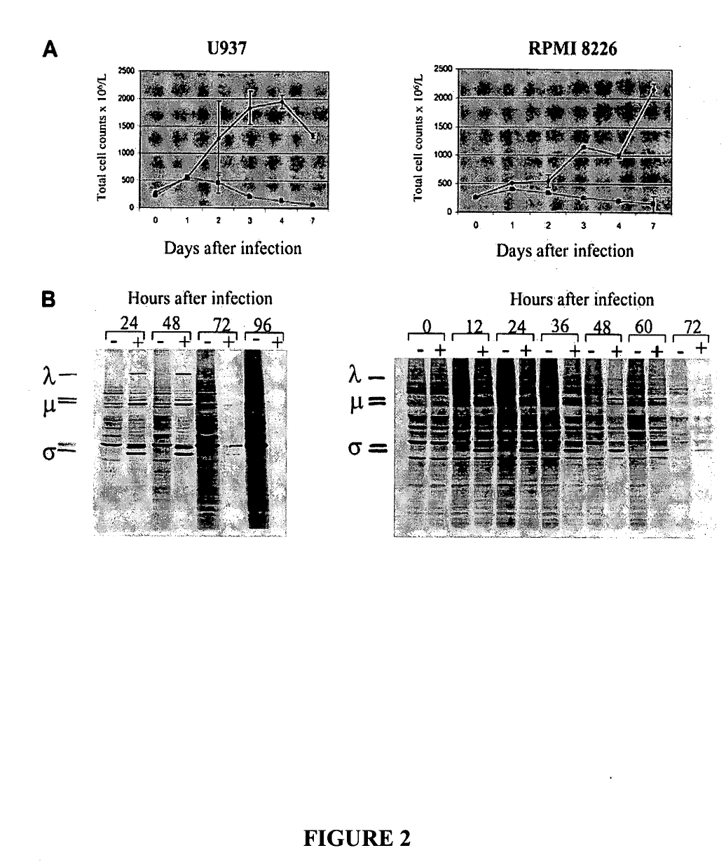

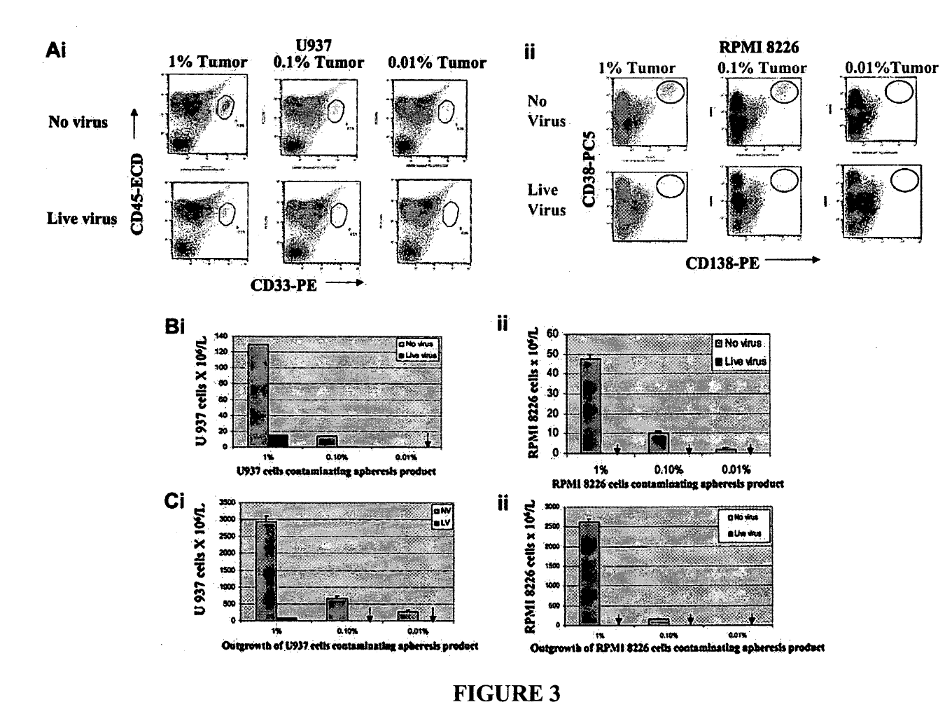

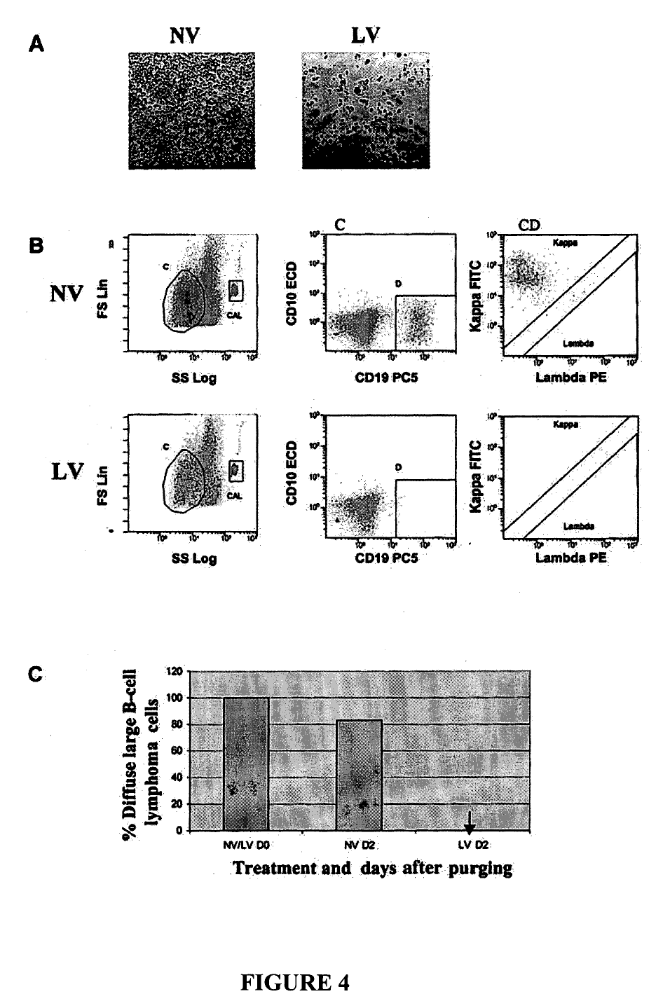

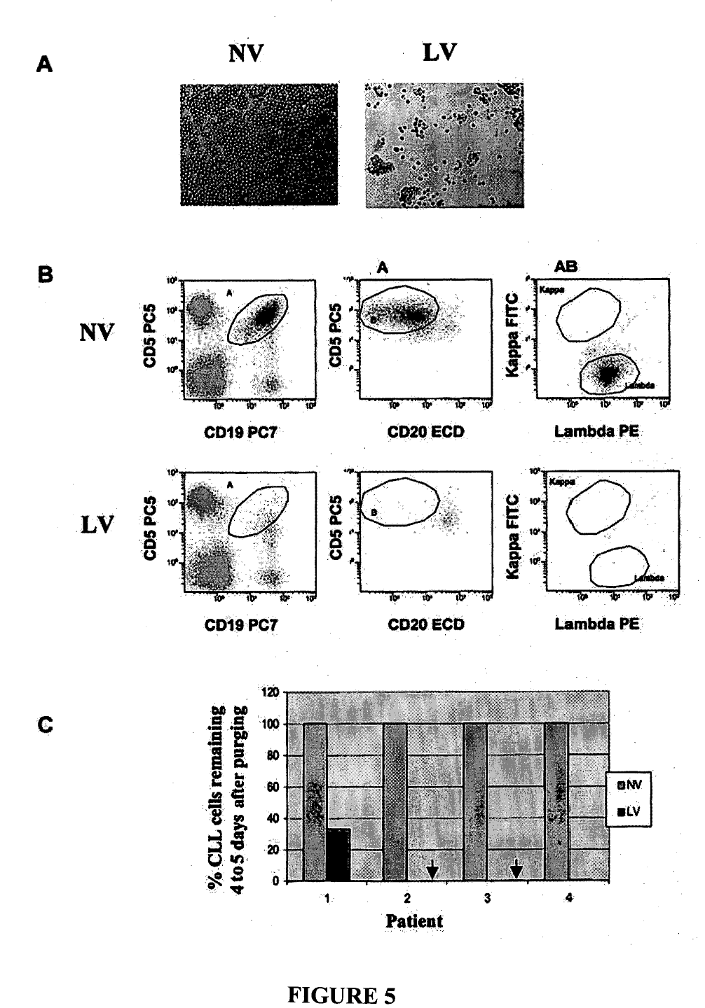

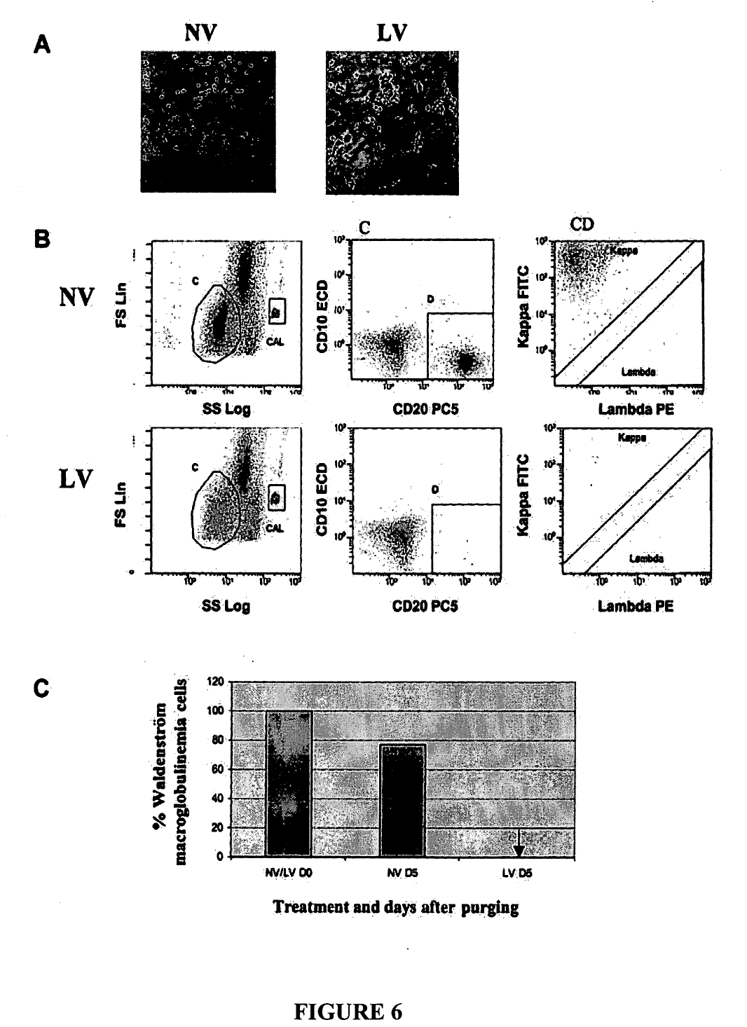

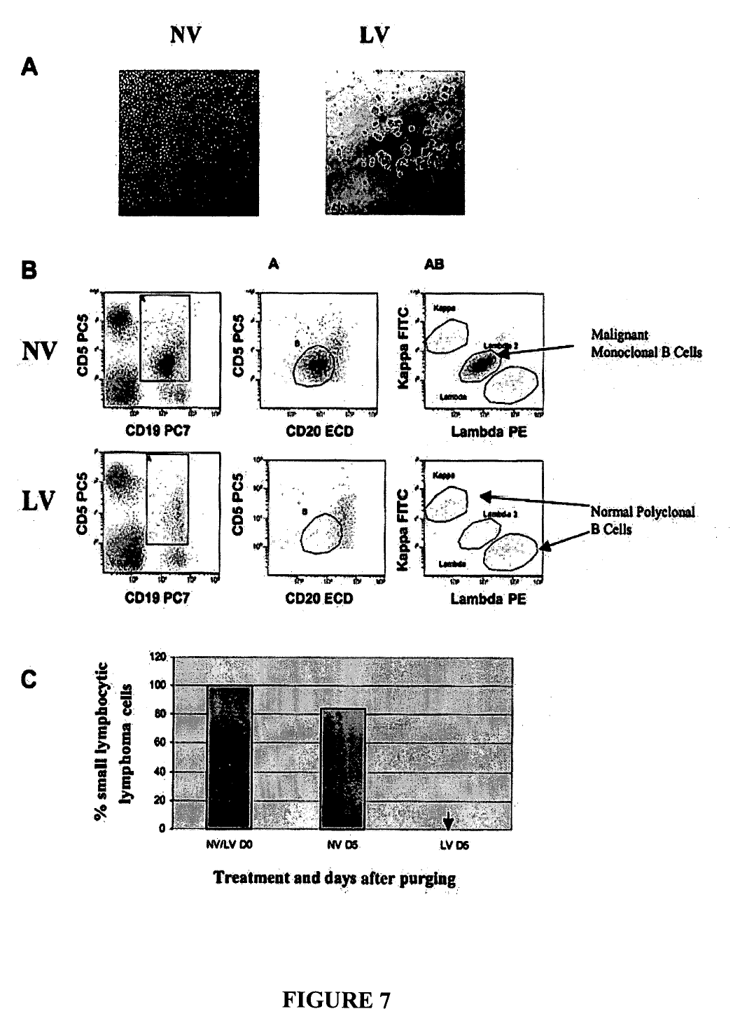

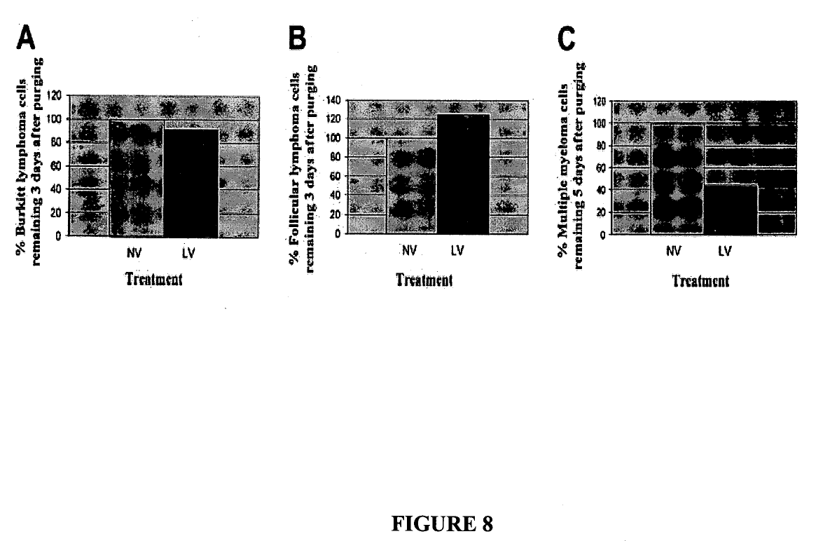

To investigate whether reovirus would affect the number and function of stem cells, positively selected (CD34⁺) stem cells were challenged with reovirus at an MOI of 40 and cultured in StemSpan medium for 5 days. As shown in FIG. 1A the number of CD34⁺CD45⁺ cells significantly increased with prolonged incubation and, at day 5, were 80-fold higher than at the start of the experiment. No significant difference between virus-treated and untreated stem cells was detected.

The preservation of the clonogenic potential of virus-treated and untreated hematopoietic progenitors was determined by culturing CD34⁺-enriched stem cells in StemSpan medium plated in methylcellulose medium. CFU-GMs, BFU-Es, and CFU-GEMMs were counted after 14 days of incubation. As shown in FIG. 1B, no differences in the clonogenic capacity of the stem cells was detected in virus-treated or untreated stem cells.

To confirm that reovirus does not replicate in growth factor-stimulated hematopoietic progenitor cells, AP cells were primed with G-CSF, challenged with reovirus, and pulse labeled with [35S]-methionine. Cell lysates were analyzed by SDS-PAGE. As depicted in FIG. 1C, no viral protein bands λ, μ, and σ could be detected at any of the time points tested, even after stimulation with G-CSF. Further, host cell protein synthesis was still evident in AP cells even at 60 hours after virus infection.

### EXAMPLE 2

### Flow Cytometric Analysis and [35S]-Methionine Labeling of Malignant Cell Lines

The susceptibility of established monocytic and myeloma cell lines to reovirus infection was tested by culturing U937 and RPMI 8226 cells in the presence (40 MOI) or absence of reovirus.

To confirm the cytopathic effect of reovirus on these cell lines samples of the cultured cells were obtained at days 0, 1, 2, 3, 4, and 7 days after virus infection and were analyzed for intact cell counts using propidium iodide. As shown in FIG. 2A, cell numbers declined after virus infection, contrasting the increase in uninfected cells. These results were confirmed over multiple experiments, and the cell counts approached zero by day 7. Residual cells seen at day 7 in FIG. 2A (left and right panels) are due to the fact that flow cytometry still counts membrane-intact but dying cells.

Replication of reovirus in susceptible cell lines was further confirmed by metabolic labeling with [³⁵S]-methionine and analysis of cell lysates by SDS-PAGE. Viral protein synthesis was evident in both cell lines tested (FIG. 2B). The appearance of λ, μ, and σ viral protein bands was seen as early as 12 hours after viral infection (FIG. 2B, right panel). Reovirus completely shut down and took over host cell protein synthesis as judged by the replacement of host cell protein bands with viral protein bands at 48 hours in the U937 cell line. These results are in contrast to the [35S]-methionine labeling data of AP cells in which the appearance of the viral protein bands were not seen at any of the time points tested.

### EXAMPLE 3

### Purging of Monocytic and Myeloma Cancer Cells in Apheresis Product

The results in Example 1 prove that exposure of hematopoietic stem cells to reovirus does not affect CD34⁺CD45⁺ cell counts or colony-forming potential of the hematopoietic progenitor cells in vitro. The monocytic and myeloma cancer cells were then mixed with apheresis product cells to result in tumor burdens of 1%, 0.1%, and 0.01% and purged with reovirus for 3 days. The purging efficacy of reovirus was evaluated using two different techniques: flow cytometry and cancer cell outgrowth following purging.

As depicted in FIG. 3Ai and 3Bi, reovirus treatment and purging for 3 days resulted in significant purging of U937 cells and complete purging at 0.01% contamination. When purged and unpurged admixed samples were recultured in RPMI medium for 6 days, no tumor regrowth was detected in the 0.1% and 0.01% contaminated samples (FIG. 3Ci). In contrast, U937 cell outgrowth was detected in all reovirus untreated samples.

Even more striking was the complete reovirus purging of RPMI 8226 myeloma cells at 1%, 0.1%, or 0.01% tumor burden as detected by flow cytometric analysis (FIG. 3Aii, Bii). No tumor outgrowth was detected when purged samples were cultured in RPMI medium for 6 days and analyzed by flow cytometry (FIG. 3Cii). CD34⁺ counts of the purged AP were analyzed by flow cytometry, assuring the purging procedure did not affect the CD34⁺ stem cells, confirming the results in FIG. 1 (data not presented).

### EXAMPLE 4

### Reovirus Successfully Purges Several Ex Vivo Human Tumor Cells from Apheresis Product

The purging ability of reovirus against 4 primary ex vivo hematopoietic and lymphoid tumors was confirmed. Tumor samples of CLL, DLBCL, Waldenstrom disease, and small cell lymphocytic lymphoma were initially treated with 40 MOI reovirus and observed for cytopathic effect. As depicted in FIGS. 4-7, significant reovirus cytopathic effect was seen. Reovirus was able to purge DLBCL, CLL, Waldenstrom disease, and SLL successfully after 2 to 5 days of incubation. FIG. 4B-C illustrates that reovirus was able to purge DLBCL cells completely after 2 days. Of the 4 different CLL patients that were tested (FIG. 5C), patients 2 and 3 exhibited complete purging at 10% contamination after reovirus treatment by days 4 and 5, respectively. Patient 4 appeared to purge completely at 1% contamination at day 4. Patient 1 appeared to be more resistant to reovirus, although tumor burden was significantly reduced by day 5. Tumors from patients with Waldenstrom macroglobulinemia and SLL were very sensitive, and complete purging at 10% tumor burden was attained 5 days after virus treatment (FIGS. 6, 7).

In contrast, as shown in FIG. 8A-B, a Burkitt lymphoma and a follicular lymphoma appeared to be resistant to reovirus infection, and no significant differences in tumor burdens between reovirus-treated and untreated samples could be seen at 1% contamination after 3 days of reovirus treatment. Reovirus was able to purge more than 50% of the myeloma cells from one patient's apheresis product (at 5% contamination) 5 days after treatment (FIG. 8C).

### EXAMPLE 5

### Pre-Treatment with Reovirus

To reduce the risk that the introduction of reovirus-purged transplant into a patient may cause reovirus infection in the patient, the patient can be vaccinated with reovirus before transplantation is performed. To determine the effect of such vaccination, a patient is pre-treated with reovirus.

The patient has breast cancer. Infectious reovirus is injected into the largest two tumors every other day for a week, and the sizes of the tumors are measured every three days afterwards. In addition, titers of circulating anti-reovirus antibodies are also monitored at the same interval. Her tumor sizes keep decreasing for a few months, then the decrease stops. At the same time, levels of anti-reovirus antibodies in her blood begin to increase.

Hematopoietic stem cells are then harvested from the patient and purged with reovirus in vitro. The patient is subject to high-dose chemotherapy and receives the purged stem cells in the transplantation surgery. Although the purged cells contain infectious reovirus as determined by a plague forming assay, the patient does not develop reovirus infection.

A number of embodiments of the invention have been described. Nevertheless, it will be understood that various modifications may be made without departing from the spirit and scope of the invention.

1. A method for transplanting a cellular composition into a mammal, comprising administering an oncolytic virus to the mammal in an amount sufficient to elicit an immune response to the virus in the mammal, and transplanting into the mammal a cellular composition that has been purged with the virus.

2. The method of claim 1 wherein the cellular composition comprises hematopoietic stem cells.

3. The method of claim 2 wherein the hematopoietic stem cells are harvested from the blood of the mammal.

4. The method of claim 2 wherein the hematopoietic stem cells are harvested from the bone marrow of the mammal.

5. The method of claim 1 wherein the cellular composition comprises a tissue, an organ or any portion of a tissue or an organ.

6. The method of claim 5 wherein the tissue or organ is selected from the group consisting of liver, kidney, heart, cornea, skin, lung, pancreatic islet cells, and whole blood.

7. The method of claim 1 wherein the virus is a reovirus.

8. The method of claim 1 wherein the virus is selected from the group consisting of adenovirus, herpes simplex virus, vaccinia virus, influenza virus and parapoxvirus orf.

9. The method of claim 1 wherein the mammal suffers from a neoplasm.

10. The method of claim 9 wherein the neoplasm is a ras-mediated neoplasm.

11. The method of claim 9 wherein the neoplasm is selected from the group consisting of lung cancer, prostate cancer, colorectal cancer, thyroid cancer, renal cancer, adrenal cancer, liver cancer, pancreatic cancer, breast cancer and central and peripheral nervous system cancer.

12. The method of claim 9 wherein the neoplasm is selected from the group consisting of Hodgkin's disease, multiple myeloma, non-Hodgkin's lymphoma, acute myelogenous leukemia, germ cell (testicular) cancers, brain tumors, and breast tumors.

13. The method of claim 9 wherein the mammal also receives a chemotherapeutic agent or radiation therapy.

14. The method of claim 1 wherein the purged cellular composition is stored in the presence of DMSO prior to transplantation.
